(19) United States
(12) Patent Application Publication
(10) Pub. No.: US 2021/0212986 A1
(43) Pub. Date: Jul. 15, 2021

(54) TONGKAT ALI EXTRACT PRODUCTION PROCESSES AND USES THEREOF

(71) Applicant: Innovus Pharmaceuticals, Inc., San Diego, CA (US)

(72) Inventor: Bassam Damaj, San Diego, CA (US)

(21) Appl. No.: 16/740,194

(22) Filed: Jan. 10, 2020

(51) Int. Cl. A61K 31/366; C07D 493/08; A61K 36/068; A61K 36/48; A61K 36/41; A61K 36/81; A61K 36/67; A61K 31/404

(52) U.S. Cl. CPC A61K 31/366; C07D 493/08; A61K 36/068; A61K 31/404; A61K 36/41; A61K 36/81; A61K 36/67; A61K 36/48

(57) ABSTRACT

Methods for isolating bioactive components from Eurycoma longifolia (Tongkat Ali) are described. In one aspect, the methods comprise concentrating a solution comprising Eurycoma longifolia extract by a continuous-flow evaporation process. Extracts of Eurycoma longifolia and compositions comprising Eurycoma longifolia extract and uses thereof are also provided. The extracts and the compositions are useful for increasing testosterone levels and supporting natural stamina, endurance and strength.

### BACKGROUND

### Field of the Disclosed Technology

The disclosed technology generally relates to a method for isolating bioactive components from Eurycoma longifolia (Tongkat Ali). In one aspect, the methods comprise preparing a Eurycoma longifolia extract by a continuous-flow evaporation process. The disclosed technology further relates to highly purified extracts comprising Eurycoma longifolia, composition comprising extracts of Eurycoma longifolia, and uses of such extracts and compositions. The compositions, in particular, are useful for increasing testosterone levels and supporting natural stamina, endurance, and strength.

### DESCRIPTION OF THE RELATED ART

Tongkat Ali, also known as Eurycoma longifolia, pasak bumi, long jack, or Malaysia ginseng, belongs to the Simaroubaceae plants. Alternative names include penawar pahit, bedara pahit, tongkat baginda, petala bumi, setunjang bumi, cay ba binh, and plaa-lai-pueak. Tongkat Ali is a slow-growing tropical rain forest plant, mainly found in Myanmar, Thailand, Malaysia, Vietnam, and China's Hainan Island.

The chemical constituents isolated from different parts of Tongkat Ali are mainly diterpenoids containing iron lignin skeleton and iron indole ketone alkaloids such as eurycomaoside, eurycolactone, eurycomalactone, eurycomanone and pasakbumin-B. In addition, it also contains biphenyl lignin, squalene derivatives, active polysaccharides, glycopeptides and various amino acid.

The roots and/or stems of Tongkat Ali (or extracts of these roots and/or stems) have been used in traditional and folk medicine either as a single herb or as part of multiple herb ingredients to treat dysentery, fever, malaria and sexual problems including male infertility. Tongkat Ali's functions relate to treating hypertension, scabies, jaundice, carbuncles, skin itching, malaria, diarrhea, mouth ulcers, headache and other diseases. Tongkat Ali also has the effect of enhancing male sexual function and treating male sexual dysfunction. For example, European Patent No. EP1952816 discloses a Tongkat Ali extract that has the effect of treating male sexual dysfunction. Malaysian Patent No. MY2006PI03783 disclosed the efficacy of Eurycoma longifolia polar organic solvent extract for promoting growth of sperm, improving sperm quality, and other aspects of the treatment of infertility. Japanese Patent No. JP2012092108 discloses that a Tongkat Ali root extract can be prepared as a medicine for external use for treating male sexual dysfunction. Chinese Patent No. CN102430038 discloses compositions containing Tongkat Ali can increase blood levels of testosterone. Additionally, recent studies have shown that this plant extract contains quassinoids, which also has good anti-tumor and anti-HIV effects.

Tongkat Ali extracts also may be useful for weight control. For example, U.S. Patent Publication No. 2007/0224300 discloses compositions comprising Tongkat Ali extracts that may be used to promote weight loss and help dieters maintain weight loss. Malaysian Patent No. 142166 discloses substances extracted from Eurycoma longifolia useful for treating obesity and diseases associated with obesity.

There are several existing methods to extract eurycomanone from Tongkat Ali. For example, CN103408564B and CN201610743803.5 disclose extraction and purification processes of eurycomanone from Tongkat Ali. However, these techniques extract insufficient biochemically-effective components, take a long time and use a lot of organic solvent. The low production capacity and high production cost make them unsuitable for large-scale production. Accordingly, there is a need for processes that more efficiently prepare Tongkat Ali extract, and that produce an extract that is enriched in eurycomanone.

### SUMMARY

To overcome prior the above-mentioned deficiencies in methods for preparing Tongkat Ali extract and to obtain extracts with higher concentrations of eurycomanone, it is an objective of one aspect of the disclosed technology to develop techniques that are simple, quick, pollution-free, suitable for large-scale production, and entail low energy consumption and low cost.

### BRIEF DESCRIPTION OF THE DRAWINGS

FIG. 1 describes a process of isolating bioactive components from Eurycoma longifolia according to one embodiment.

### DETAILED DESCRIPTION OF THE PREFERRED EMBODIMENTS

In some embodiments, Tongkat Ali extracts comprise quassinoids, which include eurycomanone, 13α,21-dihydroeurycomanone, 13(21)-epoxyeurycomanone, eurycomanol and its glycoside, eurycomaoside and its aglycone, including all their analogues and derivatives; and coumarins, which include 6-methoxycoumarin-7-O-σ-D-glycopyranoside, its other glycosides, analogues and derivatives.

In some embodiments, alternative extraction methods may be used, such as dipping extraction, percolation extraction, reflux extraction, microwave assisted extraction, ultrasonic extraction, supercritical extraction.

The extraction process may be carried out in a variety of solvents. In some embodiments, the solvent may comprise tetrahydrofuran (THF), acetonitrile, a C₁₋₆ alcohol (including but not limited to methanol, ethanol, n-propyl alcohol, isopropyl alcohol, tert-butyl alcohol, n-butyl alcohol, n-pentanol or n-hexanol), toluene, 1,4-dioxane, diethyl ether, methyl tert-butyl ether (MTBE), dimethylformamide (DMF), or dimethylacetamide. In some embodiments, the solvent may comprise water.

In some embodiments, the solvent may comprise a C₁₋₆ alcohol. In some embodiments, the solvent may further comprise water. In some specific embodiments, the solvent may further comprise 1, 2, 3, 4, 5, 6, 7, 8, 9, 10, 15, 20, 25, 30, 35, 40, 45, or 50% v/v water. In some embodiments, the solvent system may further comprise 1% to 10% v/v, 1% to 20% v/v 10% to 30% v/v, 20% to 40% v/v, 15% to 35% v/v, 25% to 35% v/v, 30% to 50% v/v water. In some embodiments, the solvent may be 95/5, 90/10, 85/15, 80/20, 75/25, 70/30, 65/35, 60/40, 55/45, or 50/50% v/v C₁₋₆ alcohol/water. In some embodiments, the C₁₋₆ alcohol may be ethanol. In some embodiments, the solvent may be 70/30% v/v ethanol/water.

The extraction process may be carried out by heating about 750 kg of plant materials with about 6000 L of one of the aforementioned solvents. The extraction process may be carried out at a solvent to plant material ratio of about 8 L/kg. In some embodiments, the extraction process may be carried out at a solvent to plant material ratio of about 0.5, 1, 2, 3, 4, 5, 6, 7, 8, 9, 10, 12.5, 15, 20, 25, 30, 40, or 50 L/kg. In some embodiments, the extraction process may be carried out at a solvent to plant material ratio in the range of 0.1 to 0.5, 0.3 to 1, 0.5 to 2, 1 to 3, 1 to 10, 2 to 4, 3 to 5, 4 to 6, 5 to 7, 6 to 8, 7 to 9, 8 to 10, 9 to 12.5, 5 to 15. 10 to 15, 12.5 to 20, 15 to 25, 20 to 30, 25 to 40, or 30 to 50 L/kg.

In some embodiments, the extraction may be performed at a temperature in the range of about 20° C. to 100° C. In some embodiments, the extraction may be performed at a temperature of about 20, 25, 30, 35, 40, 45, 50, 55, 60, 65, 70, 75, 80, 85, 90, 95, or 100° C. In some embodiments, the extraction may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

The extraction process may be carried out for an amount of time between about 0.1 to 10 hours. In some embodiments, the extraction process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, or 10.0 hours. In some embodiments, the extraction process may be carried out for an amount of time in the range of 0.1 to 0.5, 0.3 to 1.0, 0.5 to 1.5, 1.0 to 2.0, 1.0 to 3.0, 1.0 to 5.0, 1.5 to 2.5, 2.0 to 3.0, 2.5 to 3.5, 3.0 to 4.0, 3.0 to 5.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, or 8.0 to 10.0 hours, or for an amount of time greater than 10.0 hours. In some embodiments, the extraction process may be carried out for an amount of time between about 1.5 to 2.0 hours.

In some embodiments, the continuous-flow evaporation process is implemented in gas-liquid separators, liquid purifiers, liquid concentrators, desalination systems, or fractional-volatilization separation systems, which enable a stable, continuous, non-bubbling liquid flow, essentially constant surface area/volume ratio, temperature controlled, fractional volatilization of volatile/semi-volatile components in a liquid analyte or component containing sample. The fractional volatilization separator system can be utilized in small scale analytical and large scale chemical purification, concentration and desalinization applications. Continuous rapid removal of residual liquid sample can provide concentrated non-volatile component/analyte solution, and allows quick and easy washout between sequential uses with different liquid samples.

In some embodiments, the continuous-flow evaporation process may be performed at a temperature in the range of about 20° C. to 100° C. In some embodiments, the evaporation process may be performed at a temperature of about 20, 25, 30, 35, 40, 45, 50, 55, 60, 65, 70, 75, 80, 85, 90, 95, or 100° C. In some embodiments, the evaporation process may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

In some embodiments, the evaporation process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 15.0, 20.0, or 25.0 hours. In some embodiments, the evaporation process may be carried out for an amount of time in the range of 0.1 to 0.5, 0.3 to 1.0, 0.5 to 1.5, 1.0 to 2.0, 1.0 to 5, 1.0 to 10.0, 1.5 to 2.5, 2.0 to 3.0, 2.5 to 3.5, 3.0 to 4.0, 3.0 to 6.0, 3.0 to 10.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, 8.0 to 10.0, 9.0 to 15.0, 10.0 to 20.0, or 15.0 to 25.0 hours, or for an amount of time greater than 25.0 hours. In some specific embodiments, the evaporation process may be carried out for about 6 hours. In some embodiments, alternative concentration methods may be used, such as concentrating at atmospheric pressure, concentrating under reduced pressure and the like, concentrate drying, hot air drying, vacuum dried under reduced pressure, microwave (vacuum) drying, spray drying and other large-scale production and pharmaceutically acceptable drying methods.

In some embodiments, the macro-porous resins may be non-polar macroporous adsorption resin, or low-polarity resin, such as DA-201, D-IO1, LSA-20, HP-10 or AB-8 type macroporous resin. In some embodiments, the macro-porous resins may be non-polar resin, low-polarity resin or middle-polarity resin, the non-polar resin selected from XAD-4, Diaion HP-20, D101, D102, D401, D1, D2, D3, D4, HPD-100 or X-5, the low pole resin selected from D-201 HPD-300 or AB-8, and the middle polarity resin selected from XAD-6, XAD-7 or XAD-8.

In some embodiments, a purification process is performed by combining the desired eluted fraction with a second solvent, and heating at a temperature range of about 70-75° C. for about 8.0 hours to obtain a purified solution.

In some embodiments, the second solvent may comprise tetrahydrofuran (THF), acetonitrile, a C₁₋₆ alcohol (including but not limited to methanol, ethanol, n-propyl alcohol, isopropyl alcohol, tert-butyl alcohol, n-butyl alcohol, n-pentanol or n-hexanol), toluene, 1,4-dioxane, diethyl ether, methyl tert-butyl ether (MTBE), dimethylformamide (DMF), or dimethylacetamide. In some embodiments, the second solvent may comprise water. The second solvent may be the same as, or different than, the first solvent.

In some embodiments, the second solvent may comprise a C₁₋₆ alcohol. In some embodiments, the second solvent may further comprise water. In some specific embodiments, the second solvent may further comprise 1, 2, 3, 4, 5, 6, 7, 8, 9, 10, 15, 20, 25, 30, 35, 40, 45, or 50% v/v water. In some embodiments, the second solvent system may further comprise 1% to 10% v/v, 1% to 20% v/v 10% to 30% v/v, 20% to 40% v/v, 15% to 35% v/v, 25% to 35% v/v, 30% to 50% v/v water. In some embodiments, the second solvent may be 95/5, 90/10, 85/15, 80/20, 75/25, 70/30, 65/35, 60/40, 55/45, or 50/50% v/v C₁₋₆ alcohol/water. In some embodiments, the C₁₋₆ alcohol is ethanol. In some embodiments, the second solvent may be 70/30% v/v ethanol/water.

In some embodiments, the purification process may be performed at a temperature in the range of about 20° C. to 100° C. In some embodiments, the purification process may be performed at a temperature of about 20, 25, 30, 35, 40, 45, 50, 55, 60, 65, 70, 75, 80, 85, 90, 95, or 100° C. In some embodiments, the purification process may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

In some embodiments, the purification process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 15.0, 20.0, or 25.0 hours. In some embodiments, the purification process may be carried out for an amount of time in the range of 0.1 to 0.5, 0.3 to 1.0, 0.5 to 1.5, 1.0 to 2.0, 1.5 to 2.5, 2.0 to 3.0, 2.5 to 3.5, 3.0 to 4.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, 8.0 to 10.0, 9.0 to 15.0, 10.0 to 20.0, or 15.0 to 25.0 hours, or for an amount of time greater than 25.0 hours.

In some embodiments, a spraying-drying process may be performed by feeding the purified solution into a spraying-drying tank, and collecting a dried powder. The spraying-drying tank may be maintained at a temperature of about 75° C. and having an inlet air temperature of about 165° C. and an outlet air temperature of about 75° C. In some embodiments, the spraying-drying tank may be maintained at a temperature of about 45, 50, 55, 60, 65, 70, 75, 80, 85, 90, or 95° C. In some embodiments, the spraying-drying tank may be maintained at a temperature in the range of 40 to 45, 43 to 50, 45 to 55, 50 to 60, 55 to 65, 60 to 70, 65 to 75, 70 to 80, 75 to 85, 80 to 90, or 85 to 95° C., or at a temperature greater than 95° C. In some embodiments, the spraying-drying tank may have an inlet air temperature of about 135, 140, 145, 150, 155, 160, 165, 170, 175, 180, 185, 190, 195, or 200° C. In some embodiments, the spraying-drying tank may have an inlet air temperature in the range of 135 to 143, 140 to 145, 143 to 150, 145 to 155, 150 to 160, 155 to 165, 160 to 170, 165 to 175, 710 to 180, 175 to 185, 180 to 190, 185 to 195, or 190 to 200° C., or have an inlet air temperature greater than 200° C. In some embodiments, the spraying-drying tank may have an outlet air temperature of about 45, 50, 55, 60, 65, 70, 75, 80, 85, 90, or 95° C. In some embodiments, the spraying-drying tank may have an outlet air temperature in the range of 40 to 45, 43 to 50, 45 to 55, 50 to 60, 55 to 65, 60 to 70, 65 to 75, 70 to 80, 75 to 85, 80 to 90, or 85 to 95° C., or have an outlet air temperature greater than 95° C.

In some embodiments, the presence and contents of quassinoids and coumarins, including their analogues and derivatives, as such analogues and derivatives may be present in the Tongkat Ali plant material or may be a by-product generated by the production method. These components may be present in extracts, dried powder, compositions and/or pharmaceutical products, are analyzed by chromatographic processes including reversed phase high-performance liquid chromatography (HPLC) and mass spectroscopy (MS) and identified by ultraviolet, infrared, mass spectroscopies and nuclear magnetic resonance and X-ray diffraction analysis.

In some embodiments, the compositions and/or pharmaceutical products comprise a high percentage of the compound eurycomanone. In some embodiments, the compositions and/or pharmaceutical products comprise a high percentage of bioactive components of Eurycoma longifolia.

In some embodiments, a composition is provided comprising Eurycona longifolia extract. In some embodiments, the composition is formulated for oral administration. For example, the composition may be prepared as a capsule, tablet, powder, or sachet. In some embodiments, a unit dose of the composition may be one, two, three, four, or more capsules. In some specific embodiments, a unit dose of the composition may be two capsules.

In some embodiments, the composition comprises Eurycoma longifolia extract. In some embodiments, the composition is in the form of a capsule for oral administration. In some embodiments, the amount of Eurycoma longifolia extract in the composition may be about 50, 60, 70, 80, 90, 100, 125, 150, 175, 200, 225, 250, 275, 300, 350, 400, 450, or 500 mg or within a range defined by any two of the aforementioned values per capsule. Approximately 10 wt. %, 15 wt. %, 20 wt. %, 25 wt. %, 30 wt. %, 35 wt. %, or 40 wt. % Eurycoma longifolia extract present in the composition are contemplated within the scope of the invention.

In some embodiments, the composition further comprises Fenugreek seed extract. In some embodiments, the amount of Fenugreek seed extract in the composition may be about 50, 60, 70, 80, 90, 100, 125, 150, 175, 200, 225, 250, 275, 300, 350, 400, 450, or 500 mg or within a range defined by any two of the aforementioned values per capsule. Approximately 5 wt. %, 10 wt. %, 15 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, or 35 wt. % Fenugreek seed extract present in the composition are contemplated within the scope of the invention.

In some embodiments, the composition further comprises cordyceps. Cordyceps is a fungus that is known to live on certain caterpillars in the high mountain regions of China. Cordyceps may improve immunity, may have anticancer activity, may improve athletic performance, and may treat male sexual problems. In some embodiments, the amount of cordyceps in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % cordyceps present in the composition are contemplated within the scope of the invention.

In some embodiments, the composition further comprises Rhodiola rosea extract. In some specific embodiments, the composition further comprises Rhodiola rosea root extract. In some embodiments, the amount of Rhodiola Rosea extract in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt %. 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % Rhodiola rosea extract present in the composition are contemplated within the scope of the invention.

In some embodiments, the composition further comprises Ashwagandha extract. In some specific embodiments, the composition further comprises Ashwagandha root extract. In some embodiments, the amount of Ashwagandha extract in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % Ashwagandha extract present in the composition are contemplated within the scope of the invention.

In some embodiments, the composition further comprises diindolylmethane. In some embodiments, the amount of diindolylmethane in the composition may be about 10, 20, 30, 40, 50, 60, 70, 80, 90, 100, 125, or 150, or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % diindolylmethane present in the composition are contemplated within the scope of the invention.

In some embodiments, the composition further comprises black pepper extract. In some embodiments, the amount of black pepper extract in the composition may be about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or 20 mg or within a range defined by any two of the aforementioned values per capsule. Approximately 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. % 1.9 wt. %, or 2.0 wt. % black pepper extract present in the composition are contemplated within the scope of the invention.

In some embodiments the composition may comprise a mass ratio of about 4:1, 4:2, 4:3, 3:1, 2:1, 1:1, 1:2, 1:3, or 1:4 of Eurocma longfolia extract to fenugreek extract. In some embodiments the composition may comprise Rhodiola rosea root extract and ashwanganda root extract in a mass ratio of about 5:1, 4:1, 3:1: 2:1, 1:1, 1:2, 1:3, 1:4 or 1:5.

In certain embodiments, the composition may comprise vitamin B6, magnesium oxide, zinc oxide, gelatin, or water; or a combination thereof.

In some embodiments, the pharmaceutical products comprise about 400 mg of Eurycoma longifolia extract in 2 capsules. In some embodiments, the pharmaceutical products comprise about 400 mg of Eurycoma longifolia extract, about 300 mg of fenugreek seed extract, about 100 mg of Cordyceps mycelium, about 100 mg of Rhodiola rosea extract, about 100 mg of Ashwagandha extract, about 66 mg of diindolylmethane (DIM), about 14 mg of black pepper extract, about 20 mg of vitamin B6, about 100 mg of magnesium, and about 30 mg of zinc in 2 capsules. In some embodiments, the pharmaceutical products comprise about 200 mg of Eurycoma longifolia extract per capsule. In some embodiments, the pharmaceutical products comprise about 200 mg of Eurycoma longifolia extract, about 150 mg of fenugreek seed extract, about 50 mg of Cordyceps mycelium, about 50 mg of Rhodiola rosea extract, about 50 mg of ashwagandha extract, about 33 mg of diindolylmethane (DIM), and about 7 mg of black pepper extract per capsule. In some embodiments, each capsule may further comprise about 10 mg of vitamin B6, about 50 mg of magnesium, and about 15 mg of zinc.

In some embodiments, the vitamin B6 is in the form of pyridoxine hydrochloride. In some embodiments, the magnesium is in the form of magnesium oxide. In some embodiments, the zinc is in the form of zinc oxide. In some embodiments, black pepper extract is in the form of BioPerine®. In some embodiments, the Eurycoma longifolia extract is obtained from the root. In some embodiments, the Rhodiola rosea extract is obtained from the root. In some embodiments, the ashwagandha extract is obtained from the root.

In some embodiments, the pharmaceutical products comprise 400 mg of Eurycoma longifolia extract per 1230 mg of pharmaceutical product. In some embodiments, the pharmaceutical products comprise about 32.5% of Eurycoma longifolia extract. In some embodiments, the pharmaceutical products may comprise about 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, 97.5%, or 100% of Eurycoma longifolia extract. In some embodiments, the pharmaceutical products may comprise about 0.1 to 5%, 3 to 10%, 5 to 15%, 10 to 20%, 15 to 25%, 20 to 30%, 25 to 30%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70%, 67 to 75%, 70 to 80%, 75 to 85%, 80 to 90%, 85 to 95%, or 90 to 100% of Eurycoma longifolia extract.

In some embodiments, the Eurycoma longifolia extract may comprise more than about 1%, 2%, 3%, 5%, 7%, 10%, 15%, 20%, 25%, 30%, 40%, 50%, 60%, 70%, 80%, or 90% of the compound eurycomanone by weight. In some embodiments, the Eurycoma longifolia extract may comprise at least about 1%, 2%, 3%, 5%, 7%, 10%, 15%, 20%, 25%, 30%, 40%, 50%, 60%, 70%, 80%, or 90% of the compound eurycomanone by weight. In some embodiments, the Eurycoma longifolia extract prepared according to the disclosed methods may comprise about 1 to 3%, 2 to 5%, 3 to 10%, 5 to 15%, 10 to 20%, 15 to 25%, 20 to 30%, 25 to 30%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70%, 67 to 75%, 70 to 80%, 75 to 85%, 80 to 90%, 85 to 95%, or 90 to 100% of the compound eurycomanone by weight. Such extracts may comprise percentages of eurycomanone that are greater than could be obtained by prior processes

In some embodiments, the dried powder prepared according to the disclosed methods (after spray-drying) comprise more than about 5% of the compound eurycomanone by weight. In some embodiments, the dried powder prepared according to the disclosed methods may comprise more than about 1%, 2%, 3%, 5%, 7%, 10%, 15%, 2000, 25%, 30%, 40%, 50%, 60%, 70%, 80%, or 90% of the compound eurycomanone by weight. In some embodiments, the dried powder prepared according to the disclosed methods may comprise about 1 to 3%, 2 to 5%, 3 to 10%, 5 to 15%, 10 to 20%, 15 to 25%, 20 to 30%, 25 to 300%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70, 67 to 75%, 70 to 80, 75 to 85%, 80 to 90%, 85 to 95%, or 90 to 100% of the compound eurycomanone by weight.

In some embodiments, the pharmaceutical products comprise more than about 1.62% of the compound eurycomanone by weight. In some embodiments, the pharmaceutical products may comprise more than about 0.25, 0.50, 0.75, 1.00, 1.25, 1.50, 1.62, 1.75, 2.00, 2.25, 2.50, 2.75, 3.00, 3.25, 3.50, 3.75, 4.00, 4.25, 4.50, 4.75, 5.00, 5.50, 6.00, 6.50, 7.00, 10.00, 15.00, 20.00, 25.00, 30.00, 40.00, 50.00, 60.00, 70.00, 80.00, or 90.00% of the compound eurycomanone by weight. In some embodiments, the pharmaceutical products may comprise about 0.1 to 0.5%, 0.25 to 0.75%, 0.5 to 1.00%, 0.75 to 1.25%, 1.00 to 1.50%, 1.25 to 1.62%, 1.50 to 1.75%, 1.62 to 2.00%, 1.75 to 2.25%, 2.00 to 2.50%, 2.25 to 2.75%, 2.50 to 3.00%, 2.75 to 3.25%, 3.00 to 3.50%, 3.25 to 3.75%, 3.50 to 4.00%, 3.75 to 4.25%, 4.00 to 4.50%, 4.25 to 4.75%, 4.50 to 5.00%, 4.75 to 5.50%, 5.00 to 6.00%, 5.50 to 6.50%, 6.00 to 7.00%, 6.50 to 10.00%, 7.00 to 15.00%, 10.00 to 20.00%, 15.00 to 25.00%, 20.00 to 30.00%, 25.00 to 40.00%, 30.00 to 50.00%, 40.00 to 60.00%, 50.00 to 70.00%, 60.00 to 80.00%, 70.00 to 90.00%, or 90.00 to 100.00% of the compound eurycomanone by weight.

In some embodiments, the compositions and/or pharmaceutical products may comprise clinically acceptable powders, granules, capsules, pills, tablets or other forms. In some embodiments, the dried powder may be added to clinically acceptable carriers, such as tablets, pills, or granules. In some embodiments, Tongkat Ali extracts made be administered via pills, granules, oral liquid, capsules, tablets, syrups, injections, or other clinically acceptable forms.

In some embodiments, pharmaceutically acceptable carriers may include, but are not limited to: saccharose, magnesium oxide, zinc oxide, dextrin, starch, lactose, mannitol, xylitol, chitosan, chitosan, Bifidobacterium sugar, talc, sodium carboxymethylcellulose (CMS-Na), microcrystalline cellulose (MCC), silica powder, α-cyclodextrin, β-cyclodextrin, polyvinylpyrrolidone (povidone), hydroxypropyl cellulose, polyethylene glycol (PEG).

In some embodiments, dyes may be added to the solutions and/or compositions, such as iron oxide yellow and/or red iron oxide and/or titanium dioxide, for the purpose of color matching, and may be used alone or in combination with the pharmaceutically acceptable carrier.

In some embodiments, the solutions and/or compositions provided herein may comprise a pharmaceutical carrier, diluent, co-solvent, emulsifier, penetration enhancer, preservative, emollient, or a combination thereof. Acceptable carriers or diluents for therapeutic use are well-known in the pharmaceutical art, and are described, for example, in Remington's Pharmaceutical Sciences, 18th Ed., Mack Publishing Co., Easton, Pa. (1990), which is incorporated herein by reference in its entirety.

Preservatives, stabilizers, dyes, fragrances, and the like may be provided in the solutions and/or compositions. For example, sodium benzoate, ascorbic acid, benzyl benzoate, and esters of p-hydroxybenzoic acid may be added as preservatives. In addition, antioxidants and suspending agents may be used. In one or more of the contemplated embodiments, alcohols, esters, sulfated aliphatic alcohols, and the like may be used as surface active agents; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

In certain embodiments, the solutions and/or compositions may comprise sorbitol, isopropyl alcohol, propylene glycol, butylated hydroxytoluene, triethanolamine, benzyl alcohol, benzyl benzolate, PEG 40-hydrogenated castor oil, acrylate/C₁₀₋₃₀ alkyl acrylate crosspolymer, disodium EDTA, or water; or a combination thereof.

The terms “pharmaceutically acceptable carrier” or “pharmaceutically acceptable excipient,” as used herein, include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “excipient,” as used herein, refers to an inert or relatively inert substance that is added to a pharmaceutical composition to impart certain properties to the composition including, without limitation, improved or desired bulk, consistency, stability, binding ability, lubrication, disintegrating ability, etc. A “diluent” is a type of excipient.

### EXAMPLES

Materials used in isolating bioactive components from Eurycoma longifolia described herein may be made by known methods or are commercially available. It is also possible to make use of variants that are known to those of ordinary skill in this art, but are not mentioned here in greater detail. The skilled artisan, given the literature and this disclosure, is well equipped to prepare the formulations of the instant application.

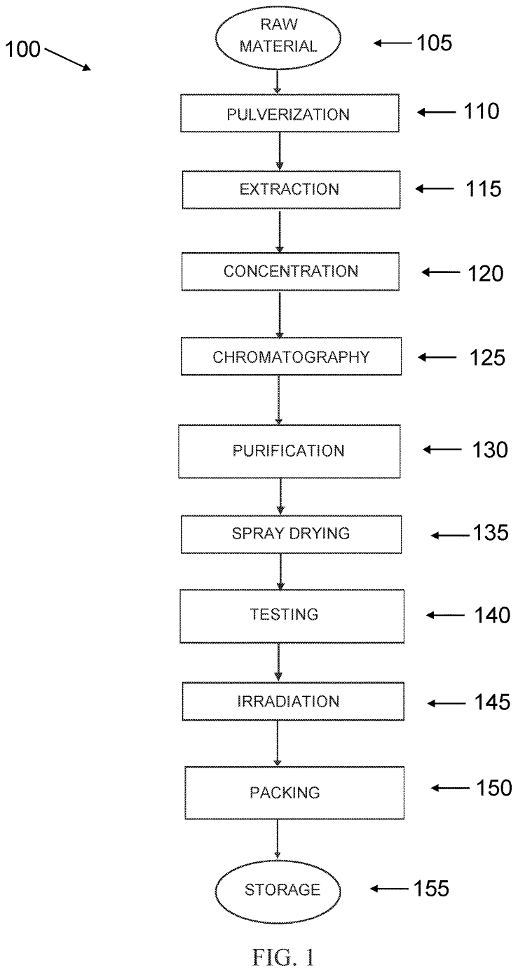

FIG. 1 describes a process 100 for isolating bioactive components from Eurycoma longifolia.

### Example 1: Processing of Raw Materials

Referring to FIG. 1, Tongkat Ali raw materials 105 are purchased from the local importer where they are clean and cut into short blocks. These blocks are spliced and thereafter pulverized in step 110 into coarse chips and weight for use in the production. No further washing and drying is needed. Materials received from the importer are subjected to sampling test using HPLC to verify eurycomanone presence.

In an embodiment for producing a 150 kg Tongkat Ali extract order, 14,250 kg of pulverized coarse chops of Tongkat Ali roots are prepared.

### Example 2A: Extraction of Processed Materials

Referring to FIG. 1, in step 115 the pulverized coarse chips of Tongkat Ali is loaded into the extraction tank and added with solvent (70% ethanol/30% water) and boiled to 70-75° C. temperature and allowed to circulate for 1.5-2.0 hours before the supernatant is drained.

In an embodiment for producing a 150 kg Tongkat Ali extract order, the process cycle data is shown in Tables 1-4 below. A total of three (3) extraction tanks were used to extract the 14,250 kg in three (3) production batches. Each production batch is a “Run” with simultaneous production using three (3) tanks. Each Run consists of three “Cycles” in reusing the Tongkat Ali materials.

TABLE 1
Conditions for Production Batch Runs

| Description | In | Out | Temp | | Duration | |
|---|---|---|---|---|---|---|
| 1) Fill TA materials | 0600 hr | 0630 hr | Ambient | | 30 | Min |
| 2) Add Solvent | 0630 hr | 0700 hr | Ambient | | 30 | Min |
| 3) Boiling | 0700 hr | 0800 hr | Reach 80° | C. | 60 | Min |
| 4) Circulation | 0800 hr | 1000 hr | 70-75° | C. | 120 | Min |
| 5) Collect Supernatant | 1000 hr | 1100 hr | 70-75° | C. | 60 | Min |
| End 1^{st} Cycle | | | | | 300 Min/5 Hrs | |
| 6) Add Solvent | 1100 hr | 1130 hr | Ambient | | 30 | Min |
| 7) Boiling | 1130 hr | 1200 hr | Reach 80° | C. | 30 | Min |
| 8) Circulation | 1200 hr | 1330 hr | 70-75° | C. | 90 | Min |
| 9) Collect Supernatant | 1330 hr | 1430 hr | 70-75° | C. | 60 | Min |
| End 2^{nd} Cycle | | | | | 210 Min/3.5 Hrs | |
| 10) Add Solvent | 1430 hr | 1500 hr | Ambient | | 30 | Min |
| 11) Boiling | 1500 hr | 1530 hr | Reach 80° | C. | 30 | Min |
| 12) Circulation | 1530 hr | 1700 hr | 70-75° | C. | 90 | Min |
| 13) Collect Supernatant | 1700 hr | 1800 hr | 70-75° | C. | 60 | Min |
| End of 3^{rd} Cycle | | | | | 210 Min/3.5 Hrs | |
| TOTAL RUN PROCESS | | | | | 720 Min/12 Hrs | |
| [TA Materials REUSED for | | | | | | |
| 2^{nd} & 3^{rd} Cycles] | | | | | | |

TABLE 2
Conditions for Production Batches

| | | | | | Solvent | | | |
|---|---|---|---|---|---|---|---|---|
| | TA | | Total | | (70%/30%) | | Supernatant | |
| CYCLE | LOAD | | Time | | IN | | OUT | |
| Tank #1 | | | | | | | | |
| 1st | 750 | kg | 5 | Hrs | 6,000 | L | 5,500 | L, |
| 2nd | Reused | | 3.5 | Hrs | 3,000 | L | 3,000 | L |
| 3rd | Reused | | 3.5 | Hrs | 3,000 | L | 3,000 | L |
| Total | 750 | kg | 12 | Hrs | 12,000 | L | 11,500 | L |
| Tank #2 | | | | | | | | |
| 1st | 750 | kg | 5 | Hrs | 6,000 | L | 5,500 | L |
| 2nd | Reused | | 3.5 | Hrs | 3,000 | L | 3,000 | L |
| 3rd | Reused | | 3.5 | Hrs | 3,000 | L | 3,000 | L |
| Total | 750 | kg | 12 | Hrs | 12,000 | L | 11,500 | L |
| Tank #3 | | | | | | | | |
| 1st | 750 | kg | 5 | Hrs | 6,000 | L | 5,500 | L |
| 2nd | Reused | | 3.5 | Hrs | 3,000 | L | 3,000 | L |
| 3rd | Reused | | 3.5 | Hrs | 3,000 | L | 3,000 | L, |
| Total | 750 | kg | 12 | Hrs | 12,000 | L | 11,500 | L |
| TOTAL BATCH | 2,250 | kg | 12 | Hrs | 36,000 | L | 34,500 | L |

TABLE 3
Weight and Volume Yields for Production Days

| | | | | | | | | Total | |
|---|---|---|---|---|---|---|---|---|---|
| Day | Hours | Batch | TA Used | | Tank #1 | Tank #2 | Tank #3 | Supernatant | |
| 10/10 | 12 Hr | 1A | 2,250 | kg | 11,500 L | 11,500 L | 11,500 L | 34,500 | L |
| 11/10 | 12 Hr | 1B | 2,250 | kg | 11,500 L | 11,500 L | 11,500 L | 34,500 | L |
| 12/10 | 12 Hr | 1C | 2,250 | kg | 11,500 L | 11,500 L | 11,500 L | 34,500 | L |
| Total 1 | | | 6,750 | kg | 34,500 L | 34,500 L | 34,500 L | 103,500 | L |
| 13/10 | 12 Hr | 2A | 2,250 | kg | 11,500 L | 11,500 L | 11,500 L | 34,500 | L |
| 14/10 | 12 Hr | 2B | 2,250 | kg | 11,500 L | 11,500 L | 11,500 L | 34,500 | L |
| 15/10 | 12 Hr | 2C | 2,250 | kg | 11,500 L | 11,500 L | 11,500 L | 34,500 | L |
| Total | | | 6,750 | kg | 34,500 L | 34,500 L | 34,500 L | 103,500 | L |
| 18/10 | 12 Hr | 3A | 750 | kg | 11,500 L | Not used | Not used | 11,500 | L |
| Total | | | 750 | kg | 11,500 L | — | — | 11,500 | L |
| TOTAL | | | 14,250 | kg | 80,500 L | 69,000 L | 69,000 L | 218,500 | L |

TABLE 4
Summary Data - Extraction

| | NO. OF | SOLVENT | | |
|---|---|---|---|---|
| | RUNS @ | USED | SUPERNATANT | YIELD |
| TA USED | 750 kg/RUN | 16:1 RATIO | YIELD | % |
| 14,250 kg | 19 | 228,000 L | 218,500 L | 95% |

### Example 2B: Alternative Method for Extraction of Processed Materials

Alternatively, referring to FIG. 1, in step 115 the pulverized coarse chips of Tongkat Ali is loaded into the extraction tank and added with solvent (90% ethanol/10% water) and boiled to 80-90° C. temperature and allowed to circulate for 3.5-5.0 hours before the supernatant is drained. In an embodiment for producing a 150 kg Tongkat Ali extract order, a total of three (3) extraction tanks were used to extract the 14,250 kg in three (3) production batches.

### Example 3A: Evaporation/Concentration of Supernatants

Referring to FIG. 1, in step 120 the supernatant collected from the extraction process is feed into the Evaporation/Concentration tank on a continuous flow and circulated for six (6) hours at 70-75° C. temperature and the total solids (thickened supernatant) is collected.

In an embodiment for producing a 150 kg Tongkat Ali extract order, the process cycle data is shown in Table 5 below. Three (3) Evaporation-Concentration tanks were used to feed the supernatant collected from the Extraction process. These were on a continuous flow as the tanks capacity are available from the draining of the thickened supernatant.

TABLE 5

| Flow | Evap #1 | | Evap #2 | | Evap #3 | | Total | |
|---|---|---|---|---|---|---|---|---|
| Supernatant In | 3,834 | L | 3,833 | L | 3,833 | L | 11,500 | L |
| Thickened | 234 | kg | 233 | kg | 233 | kg | 700 | kg |
| Supernatant Out | | | | | | | | |
| Cycle Time | 6 | hr | 6 | hr | 6 | hr | | |
| Temperature | 70-75° | C. | 70-75° | C. | 70-75° | C. | | |
| 19 Cycles IN | 72,846 | L | 72,827 | L | 72,827 | L | 218,500 | L |
| 19 Cycles OUT | 4,446 | kg | 4,427 | kg | 4,427 | kg | 13,300 | kg |

| | | | |
|---|---|---|---|
| Total Cycle Time | 108 Hr/2 Day | 108 Hr/2 Day | 108 Hr/2 Day |

### Example 3B: Alternative Methods for Evaporation/Concentration of Supernatant

Alternatively, referring to FIG. 1, in step 120 the supernatant collected from the extraction process is feed into the Evaporation/Concentration tank on a continuous flow and circulated for ten (10) hours at 70-85° C. temperature and the total solids (thickened supernatant) is collected. In an embodiment for producing a 150 kg Tongkat Ali extract order, three (3) Evaporation-Concentration tanks were used to feed the supernatant collected from the Extraction process. These were on a continuous flow as the tanks capacity are available from the draining of the thickened supernatant.

### Example 4: Column Chromatography/Separation

Referring to FIG. 1, in step 125 the thickened supernatant is feed into the column that is prefilled with non-toxic macro-porous resins/molecular sieve, and distilled water is added. Elution of impurities are at intervals during a 6 hours at ambient temperature, and the separated supernatant is collected.

In an embodiment for producing a 150 kg Tongkat Ali extract order, the process cycle data is shown in Table 6 below. Two (2) Columns were used to feed through 13,300 kg of thickened Supernatant collected from the Evaporation-Concentration process. The feed through rate is approximately 165 kg/hour and process takes two (2) days. Each column is filled with:

i. 200 kg of Macro-porous Resin/Molecular Sieve (Non Toxic)

ii. These resins are cleaned after each 1,000 kg of thickened Supernatant is feed through.

iii. The thickened Supernatant is feed in.

iv. Distilled water totaling 4,000 L is added in flushing-separation process for each 1,000 kg of thickened Supernatant.

TABLE 6
Process Cycle Data

| Flow | Column #1 | | Column #2 | | Total | |
|---|---|---|---|---|---|---|
| Feed | 6,650 | kg | 6,650 | kg | 13,300 | kg |
| Add distilled water (4,000 L | 26,600 | L | 26,600 | L | 53,200 | L |
| H₂O/1,000 kg Supernatant | | | | | | |
| Collection | 15,000 | L | 15,000 | L | 30,000 | L |

### Example 5A: Recovery/Purification

Referring to FIG. 1, in step 130 the separated supernatant is feed into the recovery tank where solvent (70% ethanol/30% water) is added to circulate for 8 hours at 70-75° C. temperature, and the purified supernatant is collected thereafter.

In an embodiment for producing a 150 kg Tongkat Ali extract order, the process cycle data is shown in Table 7 below. The separated supernatant after elution in the Column Chromatography process is then feed into the Recovery-Purification process tank. Two (2) tanks are used. Flow process rate is 600 kg per hour and process take 2 days.

TABLE 7

| Flow | Recovery #1 | | Recovery #2 | | Total | |
|---|---|---|---|---|---|---|
| Feed | 15,000 | L | 15,000 | L | 30,000 | L |
| Temperature | 70-75° | C. | 70-75° | C. | 70-75° | C. |
| Capacity 1,000 | 25 | hours | 25 | hours | 25 | hours |
| L tank with | | | | | | |
| continuous feed | | | | | | |
| at 600 L flow | | | | | | |
| rate | | | | | | |
| Thickened | 600 | kg | 600 | kg | 1,200 | kg |
| Supernatant | | | | | | |

### Example 5B: Alternative Method for Recovery/Purification

Alternatively, referring to FIG. 1, in step 130 the separated supernatant is feed into the recovery tank where solvent (60% ethanol/40% water) is added to circulate for 20 hours at 60-65° C. temperature, and the purified supernatant is collected thereafter. In an embodiment for producing a 150 kg Tongkat Ali extract order, the separated supernatant after elution in the Column Chromatography process is then feed into the Recovery-Purification process tank. Two (2) tanks are used. Flow process rate is 600 kg per hour and process take 2 days.

### Example 6: Spray Drying

Referring to FIG. 1, in step 135 the purified supernatant is feed into the spray drying tank with an inlet air temperature of 165° C. and an outlet temperature of 75° C., while the internal tank is maintain at 75° C. The flow rate through the funnel and nozzle outlet is averaging 3 kg powder per hour. The process cycle data is shown in Table 5 below.

In an embodiment for producing a 150 kg Tongkat Ali extract order, the process cycle data is shown in Table 8, below. The thickened supernatant collected from the recovery process are then feed into the spray drying machine from each production batch.

TABLE 8

| Flow rate on yield | 3 kg per hour/52.6 hours | |
|---|---|---|
| Feed | 1,200 | kg |
| Dried Powder Yield | 158 | kg |
| Ratio | 7.59:1 | (13%) |

### Example 7: Summary Analysis

In an embodiment for producing a 150 kg Tongkat Ali extract order, the summary data is shown in Table 9, below.

TABLE 9

| Flow | Start | | Add | End | | Ratio/% |
|---|---|---|---|---|---|---|
| Extraction | 14,250 | kg TA | 228,000 L | 218,500 | L | 16:1 |
| | | | Solvent | | | |
| Evaporation/ | 218,500 | L | No | 13,300 | kg | 6% |
| Concentration | | | Additives | | | |
| Supernatant | | | | | | |
| Column | 13,300 | kg | 53,200 L | 30,000 | L | 45% |
| Chromatography | | | Distilled | | | |
| | | | Water | | | |
| Recovery/ | 30,000 | L | No | 1,200 | kg | 4% |
| Purification | | | Additives | | | |
| Spray Drying - | 1,200 | kg | No | 158 | kg | 13.2% |
| Extracted | | | additives | | | |
| Powder | | | | | | |
| Net Ratio | 14,250 | kg | | 158 | kg | 90:1 |
| (Use of TA) | | | | | | |

Referring to FIG. 1, in step 140 the dried powder Tongkat Ali extract is tested using HPLC for its eurycomanone content. In an embodiment, the dried powder is found to comprise more than 5% eurycomanone level.

### Example 8: Irradiation

Referring to FIG. 1, in step 145 the dried powder Tongkat Ali extract is vacuum packed at a Controlled Facility into 1 kg bags, and loaded into 25 kg containers, sealed and sent to a controlled facility (e.g., Hunan Irradiation Center) for irradiation process using Cobalt-60 method.

### Example 9: Representative Tongkat Ali Extract Compositions

Representative formulations according to the invention are shown in Table 1 below, with the amounts for “broad,” “intermediate,” and “preferred” ranges.

TABLE 10
Representative Tongkat Ali Compositions (per capsule)

| | Broader | Narrower | Preferred |
|---|---|---|---|
| Component | Range mg | Range mg | Range mg |
| Tongkat Ali Extract | 50-500 | 100-300 | 200 |
| Fenugreek Seed Extract | 50-250 | 100-200 | 150 |
| Cordyceps | 5-100 | 25-75 | 50 |
| Rhodiola Rosea Root Extract | 5-100 | 25-75 | 50 |
| Ashwagandha Root Extract | 5-100 | 25-75 | 50 |
| diindolylmethane | 10-100 | 20-40 | 33 |
| Black pepper extract | 1-20 | 2-10 | 7 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosed technology.

1-16. (canceled)

17. A Eurycoma longifolia composition, comprising at least 10% eurycomanone by weight.

18. A composition comprising bioactive components of Eurycoma longifolia, wherein the bioactive components of Eurycoma longifolia are prepared by:
preparing an extract solution of Eurycoma longifolia plant materials;
concentrating the extract solution by a continuous-flow evaporation process; and
obtaining a thickened solution comprising the bioactive components of Eurycoma longifolia.

19. The composition of claim 18, wherein the bioactive components of Eurycoma longifolia comprise eurycomanone:

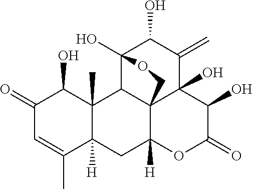

20. A pharmaceutical product comprising the composition of claim 17.

21. The pharmaceutical product of claim 20, further comprising at least one of the ingredients selected from the group consisting of fenugreek seed extract, Cordyceps mycelium, Rhodiola rosea extract, ashwagandha extract, diindolylmethane (DIM), and black pepper extract.

22. The pharmaceutical product of claim 20, further comprising at least two of the ingredients selected from the group consisting of Cordyceps mycelium, Rhodiola rosea extract, ashwagandha extract, diindolylmethane (DIM), black pepper extract, vitamin B6, magnesium, and zinc.

23. The pharmaceutical product of claim 20, further comprising fenugreek seed extract, Cordyceps mycelium, Rhodiola rosea extract, ashwagandha extract, diindolylmethane (DIM), black pepper extract, vitamin B6, magnesium, and zinc.

24. (canceled)

25. A pharmaceutical product comprising the composition of claim 19.

26. The pharmaceutical product of claim 25, further comprising at least one of the ingredients selected from the group consisting of fenugreek seed extract, Cordyceps mycelium, Rhodiola rosea extract, ashwagandha extract, diindolylmethane (DIM), and black pepper extract.

27. The pharmaceutical product of claim 25, further comprising at least two of the ingredients selected from the group consisting of Cordyceps mycelium, Rhodiola rosea extract, ashwagandha extract, diindolylmethane (DIM), black pepper extract, vitamin B6, magnesium, and zinc.

28. The pharmaceutical product of claim 25, further comprising fenugreek seed extract, Cordyceps mycelium, Rhodiola rosea extract, ashwagandha extract, diindolylmethane (DIM), black pepper extract, vitamin B6, magnesium, and zinc.

29. A method for isolating bioactive components from Eurycoma longifolia, comprising:
preparing an extract solution of Eurycoma longifolia plant materials;
concentrating the extract solution by a continuous-flow evaporation process; and
obtaining a thickened solution comprising the bioactive components of Eurycoma longifolia.

30. The method of claim 29, wherein the bioactive components of Eurycoma longifolia comprise eurycomanone.

31. The method of claim 29, wherein the plant materials comprise roots, root barks, stems, barks, leaves, flowers, fruits, or any combinations thereof, of Eurycoma longifolia.

32. The method of claim 29, wherein the plant materials are pulverized before extraction.

33. The method of claim 29, wherein preparing the extract solution comprises:
heating the plant materials with a first volume of a first solvent, at a first temperature range for a first amount of time; and
collecting a supernatant as the extract solution.

34. The method of claim 33, wherein the first solvent consists of about 70% ethanol by volume and about 30% water by volume.

35. The method of claim 34, wherein:
the first volume is about 6000 L for every 750 kg of plant materials;
the first temperature range is about 70-75° C.; and
the first amount of time is between 1.5 to 2.0 hours.

36. The method of claim 29, wherein the evaporation process occurs at a temperature range of about 70-75° C. for about 6 hours.

37. The method of claim 29, further comprising eluting bioactive components of Eurycoma longifolia, by:
passing the thickened solution through a chromatography column;
flushing an eluent through the column; and
collecting a desired eluted fraction comprising bioactive components of Eurycoma longifolia.

38. The method of claim 37, wherein:
the eluting occurs at ambient temperature;
the column is prefilled with non-toxic macro-porous resins; and
the eluent is distilled water.

39. The method of claim 37, further comprising:
combining the desired eluted fraction with a second volume of a second solvent; and
heating at a second temperature range for a second amount of time to obtain a purified solution.

40. The method of claim 39, wherein the second solvent consists of about 70% ethanol by volume and about 30% water by volume.

41. The method of claim 40, wherein:
the second temperature range is about 70-75° C.; and
the second amount of time is about 8.0 hours.

42. The method of claim 39, further comprising spray-drying the purified solution, by:
feeding the purified solution into a spraying-drying tank, the tank being maintained at a temperature of about 75° C. and having an inlet air temperature of about 165° C. and an outlet air temperature of about 75° C.; and
collecting a dried powder comprising bioactive components of Eurycoma longifolia.

43. The method of claim 42, wherein the dried powder comprises more than 5% of eurycomanone by weight.

44. The method of claim 42, further comprising sterilizing the dried powder by irradiating the dried powder with γ-rays produced by Cobalt-60 decay.
